(19) United States
(12) Patent Application Publication
(10) Pub. No.: US 2021/0338941 A1
(43) Pub. Date: Nov. 4, 2021

(54) FLUID TRANSFER DEVICES

(71) Applicant: ConceptoMed AS, Ballstad (NO)

(72) Inventors: Christian Mide, Ballstad (NO); Jimmy Gidö Schön, Ballstad (NO)

(21) Appl. No.: 17/286,560

(22) PCT Filed: Oct. 21, 2019
(86) PCT No.: PCT/EP19/78615

§ 371 Date: Apr. 19, 2021

(30) Foreign Application Priority Data
Oct. 19, 2018 (GB) 1817107.4

(51) Int. Cl. A61M 5/32

(52) U.S. Cl. CPC A61M 5/3205; A61M 2005/3128

(57) ABSTRACT

A fluid transfer device (201) comprises a body and a medical connector part (204), extending from the main body, for connection, in use, with a corresponding hub. A fluid flow path (208) extends through the body and medical connector part. A flow regulation valve is arranged in the fluid flow path in the body, for selectively controlling the flow rate of fluid through the fluid flow path. A flow control member (226) may be arranged to directly operate the flow regulation valve. A disconnection member (228) is mounted to the body and arranged to move relative to the medical connector part (204) to release, in use, the hub connected to the medical connector part. The flow regulation valve and disconnection member (228) are independently operable.

The present invention relates to fluid transfer devices, particularly those for the connection and disconnection of hubs.

The Applicant has previously devised solutions for easily disconnecting a contaminated needle from a syringe (or other fluid transfer device) using one hand as disclosed in WO 2013/164358, WO 2014/020090, WO 2015/014914 and WO 2016/162571. The Applicant's system uses a pivoting disconnecting member, e.g. lever member, to separate the needle hub from the syringe. By utilising a lever member the practitioner can, in a one handed operation, more easily disconnect the needle hub from the syringe and reduce the risk of needlestick injuries.

The Applicant has recognised that one of the problems with existing fluid transfer devices is that when the needle hub is separated from the syringe, fluid contained within the syringe may escape. This is often worse when the fluid transfer connector is supplied with fluid pressurised under gravity. At present, in order to prevent the outflow of fluid from a connector, a user must first clamp a portion of the hose connected to the fluid transfer connector with a separate, dedicated, clamp. The Applicant has appreciated that this is not ideal and that there may still be at least a small amount of fluid within the fluid transfer connector, downstream of the clamp, which may leak from the connector when the needle is disconnected.

The present invention aims to address or at least mitigate the problems outlined above, and when viewed from a first aspect provides a medical fluid transfer device comprising:

a connector part for connecting, in use, a corresponding hub;

a fluid flow path extending through the connector part; and

a flow regulation mechanism, for selectively controlling a fluid flow through the fluid flow path, wherein the flow regulation mechanism has a travel from an initial configuration to a final configuration and wherein a first portion of the travel at least partially opens the fluid flow path and wherein a second portion of the travel closes the fluid flow path and releases, in use, a corresponding hub attached to the connector part.

It will be appreciated that such a device advantageously allows a user to both selectively allow the flow of fluid through the device and close the fluid flow path whilst releasing the hub attached to the connector part. The flow regulation mechanism may initially be operated only in the first portion of travel and thus the device may initially be operated to only control the flow rate through the device. When a user wishes to disconnect an attached hub, they may operate the flow regulation mechanism through the second portion of travel to both close the fluid flow path and release the hub. Arranging the flow regulation mechanism in a manner in which the fluid flow path is closed as part of the second portion of travel which releases the hub, helps to ensure that minimal fluid escapes the fluid transfer device as the hub is released. This may help to avoid the spillage of fluids onto a work surface or patient. Further, fully closing the flow path may mean that the flow regulation mechanism can be left in its final configuration for an extended period of time, without any leakage of fluid from the device. This may be beneficial, for example, if the device were to be connected to an IV line, attached to a saline drip, which may be selectively connected to a patient.

In the second portion of travel, the flow regulation mechanism may close the fluid flow path and release the hub simultaneously. However, this may result in at least some fluid leaking out of the connector part as the hub is released. In a preferred set of embodiments, the flow regulation mechanism is arranged such that in the second portion of travel, once the flow regulation mechanism has fully closed the fluid flow path, the flow regulation then releases the hub attached to the connector part. The Applicant has recognised that this arrangement, whereby the fluid flow path is closed prior to release of the hub, may help to ensure that little, or no, fluid escapes the fluid transfer device as the hub is released. Of course, there may be a small amount of fluid residue downstream of the flow regulation mechanism which may still escape the device, however depending on the size of the device this may only be minimal. This may help to avoid wastage of the fluid being transferred by the device and also prevent the fluid from leaving the device and contaminating a user or surrounding work surfaces. Closing the flow path prior to release of the hub may be particularly beneficial when the fluid transfer device is supplied with pressurised fluid as it may reduce the amount of fluid leakage more significantly.

In a set of embodiments, in the initial configuration the fluid flow path is blocked completely. In some embodiments, the first portion of travel fully opens the fluid flow path. Such a set of embodiments ensures that a maximum fluid flow through the device can be achieved through operation of the flow regulation mechanism. In a set of embodiments, across the first portion of travel, the fluid flow path is gradually opened to increase the flow rate therethrough from a minimum to a maximum flow rate. The minimum flow rate may be a zero flow rate. As will be appreciated, in such a set of embodiments, the position of the flow regulation mechanism in the first portion of travel will determine the flow rate through the device.

In a set of embodiments, the device comprises a plurality of markings, indicative of flow rate through the device, against which an indicator part of the flow regulation mechanism may be aligned. Such a set of embodiments may allow a user to move the flow regulation mechanism to a position in which the part of the flow regulation aligns with one of the plurality of markings indicative of the flow rate through the device which they wish to achieve. For example, a user may wish to only allow 10 ml/hour through the device, and so they may move the flow regulation mechanism to a position in which the indicator part aligns with the relevant marking.

The flow regulation mechanism may comprise any arrangement capable of both controlling the fluid flow through the device and capable of releasing the hub. This may be achieved using a single member. However, in some embodiments, the flow regulation mechanism comprises a control member, arranged to control the fluid flow through the fluid flow path, and a disconnection member arranged to release, in use, the corresponding hub attached to the connector part. In the initial configuration of the flow regulation mechanism, the control member may have an initial control position and the disconnection member may have an initial connection position. In the final configuration, the control member may have a final control position and disconnection member may have a final disconnection position, i.e. a position which releases a hub from the device.

The control member and/or disconnection member may comprise one or more of a slider, lever, arm, sleeve, or any other member that can be movably mounted to the device. In some preferred embodiments, at least one of the control member and/or disconnection member comprises a pivotally mounted lever member. The Applicant has recognised that a pivotally mounted lever may be advantageous as it may amplify the force applied by a user. This may be beneficial when releasing the hub from the connector part. The disconnection member, or at least a portion thereof, may comprise a wedge-shaped portion arranged to release the hub from the connector part.

In a set of embodiments, the flow regulation mechanism is arranged such that across the first portion of travel only the control member is moved from its initial control position to an intermediate control position. In a set of embodiments, across the second portion of travel both the control member and disconnection member are moveable towards their respective final control position and final disconnection position. In such a set of embodiments, the control member may be arranged to drive movement of the disconnection member during the second portion of travel, such that the control member and disconnection member move together in unison. Such a set of embodiments may advantageously only require a user to apply a force, e.g. by gripping, to the control member to both control the flow through the device and release the hub. This Applicant has recognised that this may facilitate one-handed operation.

The disconnection member may be free to move between its initial connection position, i.e. a position in which a hub is connected and the disconnection member does not provide a force to release it, and its final disconnection position. However, in an alternative set of embodiments, the disconnection member is arranged such that when moved out of its initial connection position towards its final disconnection position, the disconnection member is resiliently biased back towards the initial connection position. As will be appreciated by those skilled in art, in such embodiments in order to move the disconnection member, the resilient bias must be overcome by a user. This may help to prevent accidental operation of the lever member which may undesirably result in disconnection of an attached hub. Further, resiliently biasing the disconnection member towards its initial connection position may, in some embodiments, help to ensure that the hub remains firmly connected to the connector part. In some embodiments, the device may comprise a spring member to provide the resilient bias. In some other embodiments the disconnection member may have its own resilient bias. For example, the disconnection member may deform elastically, at least to a degree, when a user presses on the disconnection member, such that when the user removes the pressing force, the disconnection member may return to its original form and hence move back to its original position.

In a potentially overlapping set of embodiments, the disconnection member is held in at least one of, preferably both of, the initial connection and/or final disconnection positions. The disconnection member may be held in the initial connection position for example by a resilient bias. However, in a potentially overlapping set of embodiments, the device comprises at least one further locking arrangement for holding the disconnection member stable in at least one of the initial connection or final disconnection positions. Such a locking arrangement may comprise, for example, a protrusion on the disconnection member which engages with a recess on another part of the device in at least one of the initial and/or final positions. In addition, or alternatively, the disconnection member may be shaped to fit around, or engage with, another part of the device in order to hold it in position. As will be appreciated by those skilled in the art, such a locking arrangement must first be overcome by a user before the disconnection member can be moved. This may, for example, require a user to manually release the locking arrangement, or require the user to apply at least a threshold force to the disconnection member in order to overcome the locking arrangement.

In a set of embodiments, the flow regulation mechanism is arranged to be held stable in at least one intermediate configuration across the travel, e.g. across the first and/or portion of travel, so as to selectively control the flow. This will allow a user to selectively adjust the flow rate through the device. This may be achieved in a variety of different ways depending on the form of the flow regulation mechanism. In embodiments comprising a control member, preferably the control member is arranged so as to rest, without any force from a user, in a fixed position including: at least one of the initial control position, the final control position, and in at least one position therebetween. This may allow a user to adjust the flow rate using the flow control member and release any force applied thereto with the flow rate being maintained. This may advantageously allow a user to leave the device unattended for extended periods of time whilst achieving a constant flow rate therethrough. This may be useful, for example, if the fluid transfer device is used as part of an IV line which is connected to a patient for an extended period of time. In a further set of embodiments, the flow regulation mechanism is arranged so as to be stable in a plurality of positions across the travel, thereby allowing a plurality of different flow rates to be selected by a user.

In a set of embodiments, the flow regulation mechanism comprises a lock arranged to hold the control member in the fixed position. The lock may comprise any suitable arrangement which holds the two parts in a fixed spatial relationship to one another. In a set of embodiments, the lock comprises a plurality of detents on at least one of the control member or disconnection member arranged to interact with a one or more features on the other of the control member or disconnection member. Such an arrangement may hold the control member and disconnection member in a fixed relationship, yet permit adjustment of their relative positions. In a potentially overlapping set of embodiments, the lock comprises a release arrangement which must be released by a user before the control member can be moved relative to the disconnection member. Such a set of embodiments may advantageously prevent the control member from being inadvertently operated which would result in adjustment of the flow rate through the flow device which could be fatal in certain situations.

Once the flow regulation mechanism has been moved through its travel to its final configuration resulting in the fluid flow path being closed and the hub being released from the tip, at least part of the flow regulation mechanism may be moveable back through its travel towards the initial configuration. Accordingly, in a potentially overlapping set of embodiments, at least part of the flow regulation mechanism may be moveable back from its final configuration towards its initial configuration to at least partially open the fluid flow path. As will be appreciated by those skilled in the art, this may allow a user to re-open a fluid flow through the device after the hub has been released therefrom. This may allow, for example, a user to drain fluid through the device, e.g. if it is required that the device is at least partially flushed following use. In embodiments comprising a control member and disconnection member, the above described function may be achieved by leaving the disconnection member in its disconnection position, corresponding to its position in the final configuration of the flow regulation mechanism, and moving the control member back towards its initial position relative to the disconnection member.

The flow regulation mechanism may comprise any suitable means for controlling the fluid flow through the device. For example, the flow regulation mechanism may deform part of the device, e.g. a section of flexible tubing, to inhibit the flow of fluid therethrough. In a set of preferred embodiments, the flow regulation mechanism comprises a valve arranged to control the fluid flow. In embodiments comprising a flow control member, the flow control member may be arranged to directly operate the valve.

In a set of embodiments, the connector part is a medical connector part. In a further set of embodiments, the medical connector part conforms to the requirements of one of the IS 80369 series of small-bore connector standards. The aim of this series of standards is to prevent misconnections between fluid transfer lines for different clinical uses, e.g. between enteral feeding tubes and IV lines. ISO 80369-1:2010 specifies the health fields in which fluid transfer connectors are intended to be used. These healthcare fields of use include, but are not limited to, applications for: breathing systems and driving gases; enteral and gastric; urethral and urinary; limb cuff inflation; neuraxial devices; intravascular or hypodermic. In a preferred set of embodiments the connector part conforms to one of ISO 80369-7 (Luer Fit), ISO 80369-3 (ENFit) or ISO 80369-6 (NRFit). The Applicant has recognised that providing the fluid transfer device with a connector part which conforms to one of the above ISO standards may help to prevent the misconnection of hubs which are non-compliant with the connector part. For example, it may prevent a Luer fit hub from being connected to a fluid transfer device having an NRFit connector part. This may help to avoid inadvertently administering an incorrect fluid to a patient.

In a set of embodiments, the connector part comprises a fluid transfer tip. The fluid transfer tip is preferably of the type which creates a fluid-tight connection with the hub attached thereto. The fluid transfer tip may take any suitable form for creating such a fluid-tight connection. For example, the fluid transfer tip may comprise a cylindrical tip with a rubber o-ring, extending around its circumference, arranged to create a fluid-tight seal with a hub attached thereto. In a set of embodiments, the fluid transfer tip is tapered for creating a friction fitting, in use, with a hub attached thereto. The Applicant has recognised that a tapered fluid transfer tip may remove the need to have a separate seal, such as an o-ring mentioned above, which may make the device more simplistic and thus easier to manufacture. Such a tapered tip is also a feature of many of the ISO 80369 standards referred to above.

In some embodiments, the flow regulation mechanism, for example the disconnection member where provided, is arranged to push against the hub and thereby release the connection. For example, the flow regulation mechanism may be arranged to release the connection by moving along a surface of the connector part to push away the corresponding hub. For example, the flow regulation mechanism may be arranged such that during operation a part of the flow regulation physically moves along the surface of the connector part. As an alternative example, the flow regulation mechanism may comprise a wedge-shaped disconnection member arranged such that a front surface of the wedge-shaped disconnection member moves along the surface of the connector part to push against the hub.

In embodiments comprising a tapered tip, the flow regulation mechanism, for example the disconnection member where provided, may be arranged to release an attached hub by moving at least partially along the tapered tip in order to advance the hub along the tapered tip and release the friction fitting.

The connector part may comprise an engagement part that positively engages with the hub, for example with outer threads on the hub. The engagement part may include a snap-fit connection, latch means, gripping fingers etc. that positively engage, i.e. grip, a hub when it is connected. This may be particularly suitable for high pressure fluid connection, e.g. when transferring or collecting more viscous fluids. In a set of embodiments, the engagement part comprises a collar extending at least partially around the fluid transfer tip. In a preferred set of embodiments, the collar extends 360° around the fluid transfer tip. In another set of embodiments, the collar comprises at least one engagement feature for engaging, in use, with a corresponding engagement feature provided on a hub attached to the connector part. The collar and engagement feature may be compliant with any one of the ISO standards referred to above. In a preferred set of embodiments, the at least one engagement feature comprises an internally threaded portion. Of course the internally threaded portion may only extend around part of the collar.

In a set of embodiments, the collar comprises a first segment and second segment, wherein the second segment is arranged to be moved by the flow regulation mechanism from an initial position, in which it is arranged to engage, in use, with a hub attached to the connector part, to a final position in which it is disengaged from the hub. A segmented collar with at least one part which is arranged to move to disengage from the hub may facilitate the removal of hubs comprising an external thread, without requiring rotation of the hub. In a set of embodiments, the second segment of the collar is integrally provided with the flow regulation mechanism. For example, in embodiments comprising a disconnection member, the second segment of the collar may be integrally provided with the disconnection member, e.g. the second segment may extend from a forward portion of the disconnection member.

In a set of embodiments, the device further comprises an integral fluid chamber in fluid communication with the connector part. This may form, for example, a syringe. Such a device may advantageously allow a user to set the flow rate out of the device to ensure, for example, that a fluid isn't administered to, or drawn from, a patient too quickly.

In an another set of embodiments, the device comprises a second connector part, in fluid communication with the connector part. The device could equally comprise one or more further connector parts. The plurality of further connector parts may allow a plurality of different fluids to combine and pass through the fluid transfer device. As will be appreciated by those skilled in the art, the flow regulation mechanism will regulate the combined flow of fluid entering the fluid transfer device through the second and further connector part(s). The second and any further connector part(s) may take any suitable form which allows connection of a further component. The second and any further connector part(s) may, optionally, conform to the requirements of one of the IS 80369 series of small-bore connector standards. As will be appreciated by those skilled in the art, such a set of embodiments provides a fluid transfer device in the form of a connector which may be connected between two different components. For example, the second and any further connector part(s) may be connected to flexible tubing provided with a source of fluid, e.g. an IV line connected to a saline solution. In another set of embodiments, the fluid transfer device comprises an integral fluid transfer hose in fluid communication with the connector part.

As will be appreciated by those skilled in the art, the fluid transfer device may be arranged to allow fluid to flow out through the device, i.e. out through the connector part, and in addition or alternatively, it may also be arranged to allow fluid to flow in through the device, i.e. in through the connector part. The direction of flow through the device may depend on its application, for example if it is being used to administer fluid to a patient, or if it is being used to draw a fluid from a patient. Similarly, the direction of flow through the device may depend on how the device is connected in a system, i.e. whether the connector part is connected to a source of fluid, or whether the source of fluid is provided elsewhere on the device, e.g. on the second connector part where provided.

In a set of embodiments, the fluid transfer device comprises a main body from which the connector part extends and to which the flow regulation mechanism is moveably mounted. In embodiments wherein the flow regulation mechanism comprises a valve, the valve is preferably integrally provided within the main body.

The Applicant has recognized that a device having integral means for disconnection of a connected hub, together with integral means for controlling the flow of fluid through the device which can be independently controlled from the means for disconnection is novel and inventive in its own right and thus when viewed from a second aspect the present invention provides a fluid transfer device comprising:

a body;

a medical connector part, extending from the main body, for connection, in use, with a corresponding hub;

a fluid flow path extending through the body and medical connector part;

a flow regulation mechanism or valve arranged in the fluid flow path in the body, for selectively controlling the flow rate of fluid through the fluid flow path;

a disconnection member mounted to the body and arranged to move relative to the medical connector part to release, in use, the hub connected to the medical connector part; and

wherein the flow regulation valve and disconnection member are independently operable.

As will be appreciated by those skilled in the art, the ability to control fluid flow through the device, and disconnection of a hub from the device is integrally provided with the device and so further medical components, such as a hose clamp, are not required in order to achieve these functions. The device may therefore simplify a procedure being carried out by a practitioner as they can perform multiple tasks using a single device. A user may operate the flow regulation mechanism to obtain a desired flow rate through the device and, irrespective of the state of the flow regulation valve, may also independently operate the disconnection member to release the hub.

In one or more embodiments, the flow regulation valve is independently movable between an initial configuration and a final configuration while the disconnection member remains in an initial connection position. This means that the flow rate can be varied without disconnecting a hub.

In one or more embodiments, the flow regulation valve has a travel from an initial configuration, in which the flow regulation valve is arranged to at least partially open the fluid flow path, to a final configuration, in which the flow regulation valve is arranged to close the fluid flow path. The flow rate may therefore be varied by opening/closing the fluid flow path.

In one or more embodiments, the position of the flow regulation valve in the travel determines the flow rate through the device across the travel, such that the fluid flow path may be gradually closed to decrease the flow rate therethrough from a maximum to a minimum flow rate.

In one or more embodiments, the flow regulation valve is arranged so as to be stable in a plurality of positions across the travel. The flow regulation valve may move continuously between its initial configuration and final configuration.

In one or more embodiments, the flow regulation valve is movable back from the final configuration towards the initial configuration to at least partially open the fluid flow path. This movement may take place in unison with the disconnection member.

In one or more embodiments, the disconnection member is moveable relative to the medical connector part from an initial connection position in which, in use, a corresponding hub may be connected to the medical connector part, to a final disconnection position in which, in use, the disconnection member acts to release the hub connected to the medical connector part.

In one or more embodiments, the movement of the disconnection member towards its final disconnection position is arranged to drive movement of the flow regulation valve towards its final configuration, such that the flow regulation valve and the disconnection member move together in unison. This means that the fluid flow path is always shut off during the disconnection process, resulting in dry disconnection without any fluid escape.

In one or more embodiments, the disconnection member is independently movable between its/an initial connection position and its/a final disconnection position while the flow regulation valve is in its/a final configuration. This means that the fluid flow path can remain closed while the disconnection member is moved independently to connect or release a hub in use. One hub may be released and another one connected before the flow regulation valve is moved to open the fluid flow path again.

In one or more embodiments, as described above, the medical connector part comprises a fluid transfer tip.

In one or more embodiments, as described above, the fluid transfer tip is tapered and the disconnection member is arranged to release an attached hub by moving at least partially along the tapered tip in order to advance the hub along the tapered tip and release a friction fitting.

In one or more embodiments, the disconnection member comprises a shoulder arranged to move forwards along the tapered tip only when the disconnection member moves towards its/a final disconnection position. It is preferable that the attached hub is only actively released from the friction fitting when the disconnection member moves into its/a final disconnection position.

In one or more embodiments, as described above, the medical connector part comprises a collar extending at least partially around the fluid transfer tip and arranged in use to positively engage with a hub.

In one or more embodiments, as described above, the collar comprises a first segment and a second segment, wherein the second segment is arranged to be moved by the disconnection member from an initial engagement position, in which it is arranged to engage, in use, with the hub attached to the connector part, to a final disengagement position in which it is disengaged from the hub.

In one or more embodiments, as described above, the second segment of the collar is integrally provided with the disconnection member.

In one or more embodiments, as described above, the second segment of the collar comprises an internally threaded portion.

In one or more embodiments, as described above, the device further comprises an integral fluid chamber in fluid communication with the medical connector part. The fluid transfer device is preferably a syringe.

In one or more embodiments, as described above, the disconnection member comprises a pivotally mounted lever member.

In one or more embodiments, as described above, the device may further comprise a flow control member arranged to directly operate the flow regulation valve. Preferably the flow control member comprises a pivotally mounted lever member. The disconnection member and the flow control member may be pivotally connected together. The disconnection member and the flow control member may share a common pivot point.

In one or more embodiments, the disconnection member is arranged beneath the flow control member such that movement of the disconnection member towards its/a final disconnection position always drives movement of the flow control member so as to cut off the flow rate of fluid through the fluid flow path. This ensures dry disconnection and prevents fluid spill.

Similarly to the first aspect of the invention, the device may comprise an integral fluid chamber thus forming a device such as a syringe, or alternatively the device may comprise a second connector part in fluid communication with the medical connector part for connection to a further component, e.g. a fluid transfer hose. Irrespective, as will be appreciated by those skilled in the art, the flow regulation valve and disconnection member are integrally provided with the device and are part of, or mounted to, the body of the device.

Features of previous embodiments of the present invention may, where appropriate, also be applied to this second aspect of the invention.

As will be appreciated, the hub may provide the connection point of any one of a large number of different medical components. For example, the hub may be part of a needle assembly or fluid transfer hose. Similarly, the hub may take any suitable form for attachment with the connector part. For example, it may be a female hub having a tapered internal surface for use with embodiments comprising a tapered tip. Alternatively, the hub may have a male profile for engagement with a connector part having a female configuration. In at least some embodiments, the hub may have corresponding engagement features on an outside surface thereof positioned to be engaged by engagement features on the connector part. The hub may conform to any one of the ISO 80369 standards referred to above.

Some preferred embodiments of the present invention will now be described, by way of example only, and with reference to the accompanying drawings, in which:

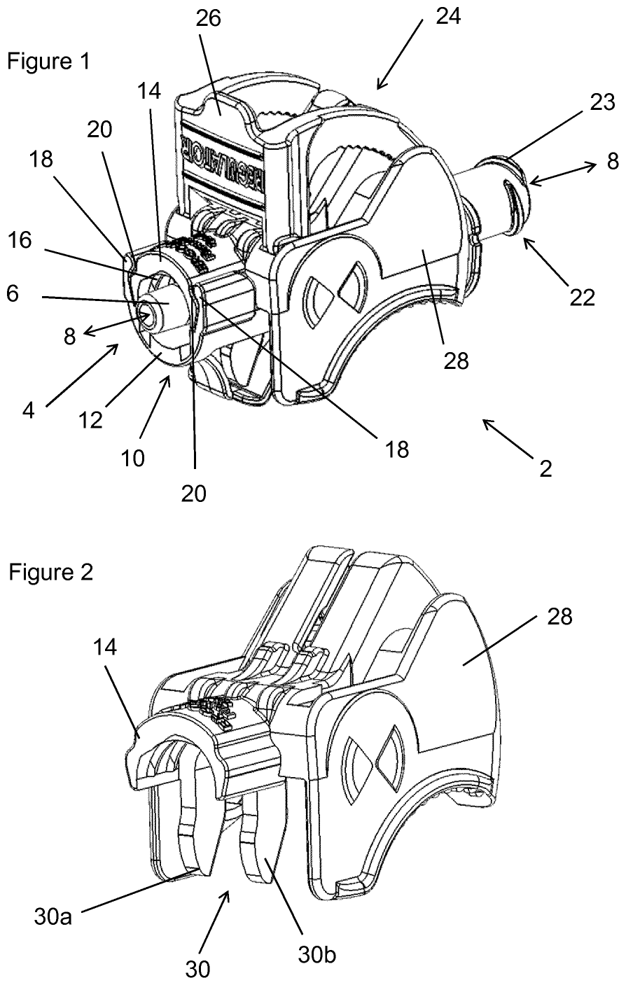

FIG. 1 shows a perspective view of a fluid transfer device in accordance with an embodiment of the present invention, with the flow regulation mechanism in its initial configuration;

FIG. 2 shows a perspective view of the disconnection member, of the connector seen in FIG. 1, in isolation;

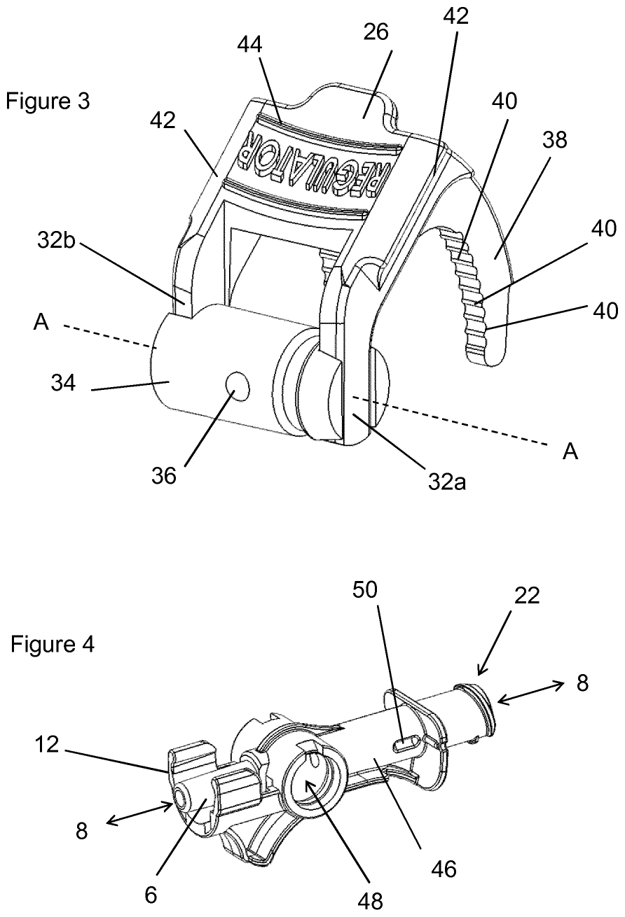

FIG. 3 shows a perspective view of the flow control member, of the connector seen in FIG. 1;

FIG. 4 shows a perspective view of the main body of the fluid transfer device seen in FIG. 1;

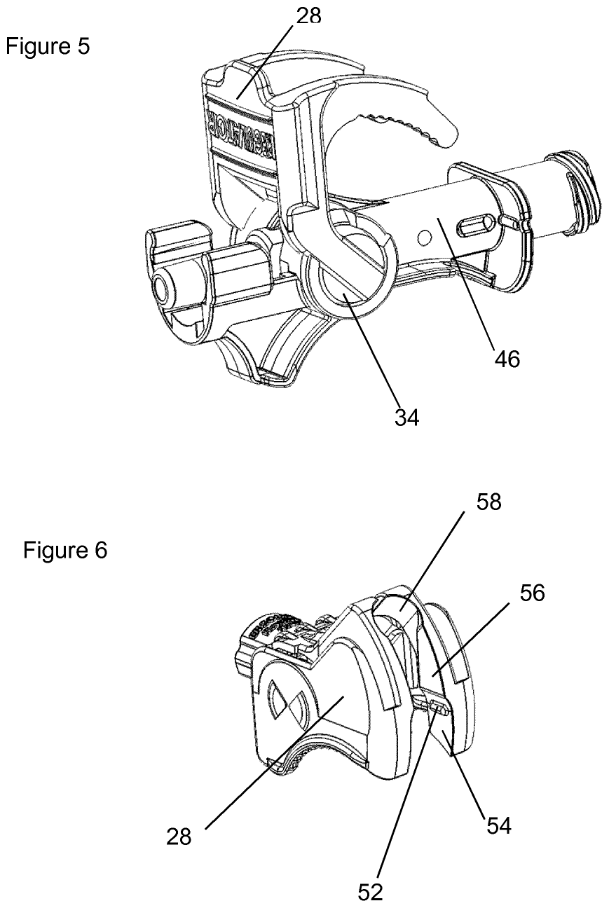

FIG. 5 shows a perspective view of the main body and flow control member assembled together;

FIG. 6 shows a perspective view, when viewed from the rear, of the disconnection member;

FIG. 7 shows a cut-away perspective view, when viewed from the rear, of the fluid transfer device seen in FIG. 1;

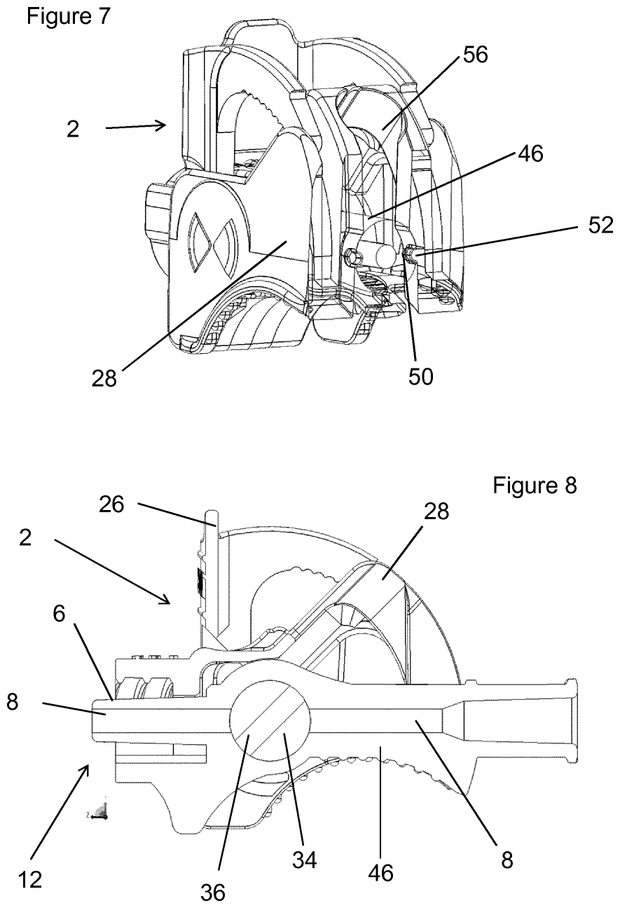

FIG. 8 shows a cross sectional view through the fluid transfer device seen in FIG. 1;

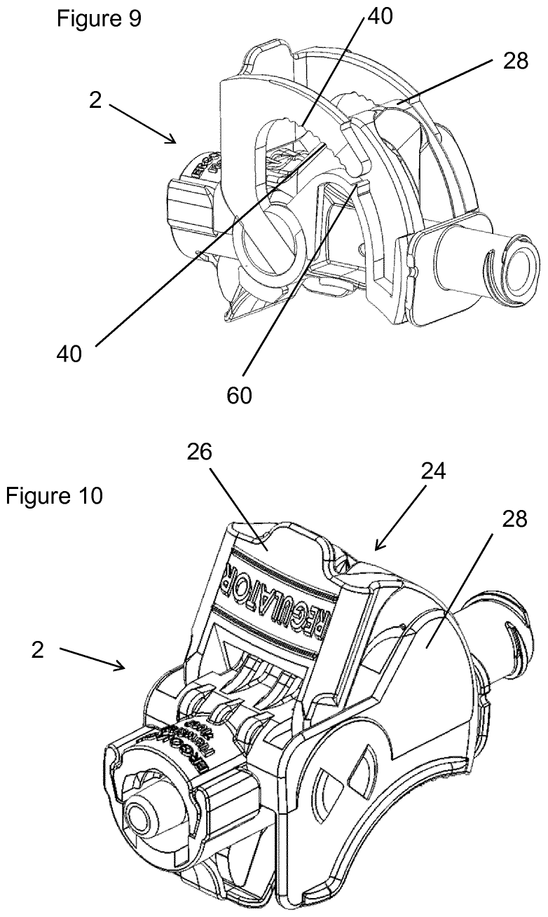

FIG. 9 shows a cut-away perspective view of the fluid transfer device seen in FIG. 1;

FIG. 10 shows a perspective view of the fluid transfer device seen in FIG. 1 with the flow regulation mechanism position in an intermediate configuration;

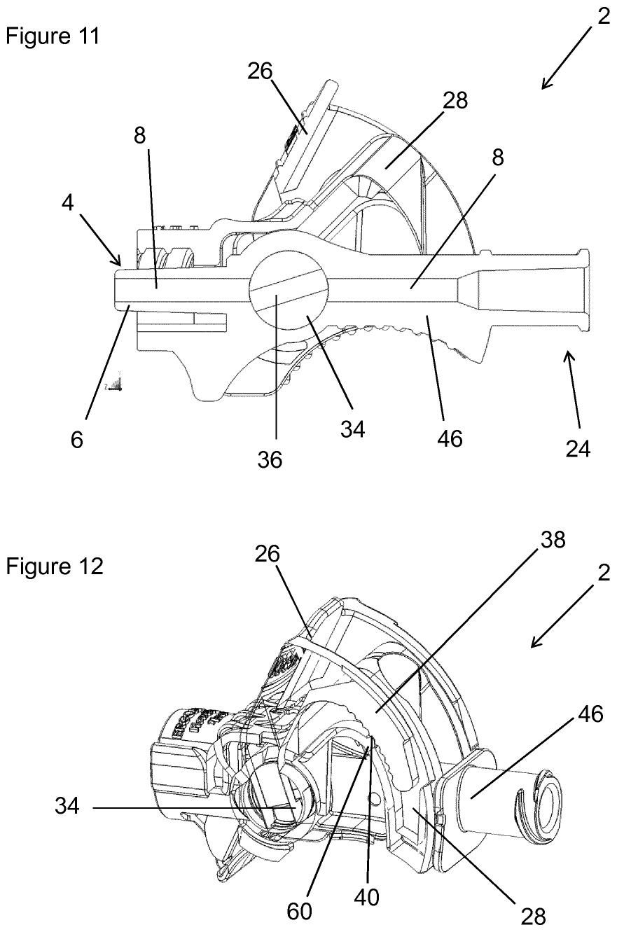

FIG. 11 shows a cross-sectional view of the fluid transfer device in the configuration seen in FIG. 10;

FIG. 12 shows a cut-away perspective view of the fluid transfer device in the configuration seen in FIG. 10;

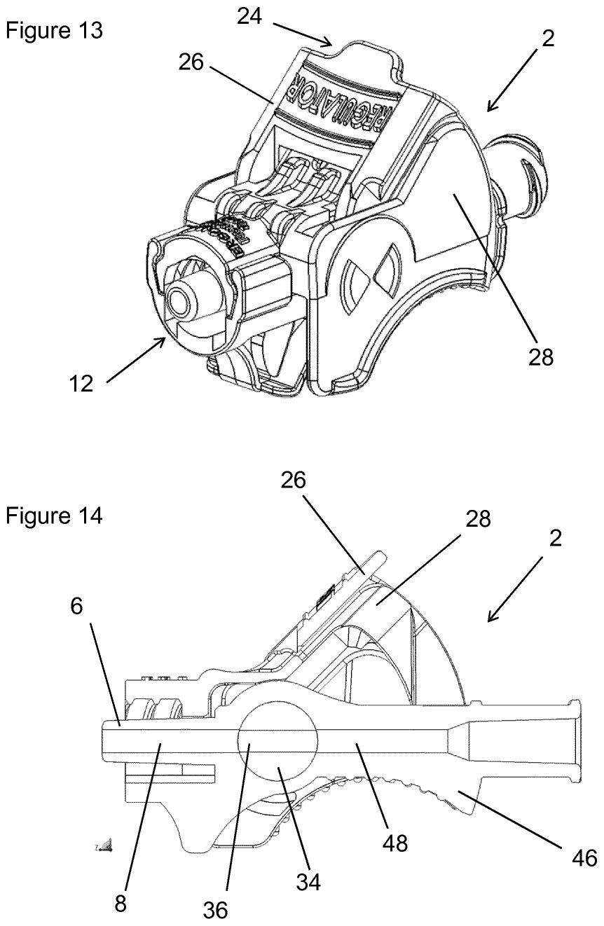

FIG. 13 shows a perspective view of the fluid transfer device seen in FIG. 1 with the flow regulation mechanism moved through its first portion of travel;

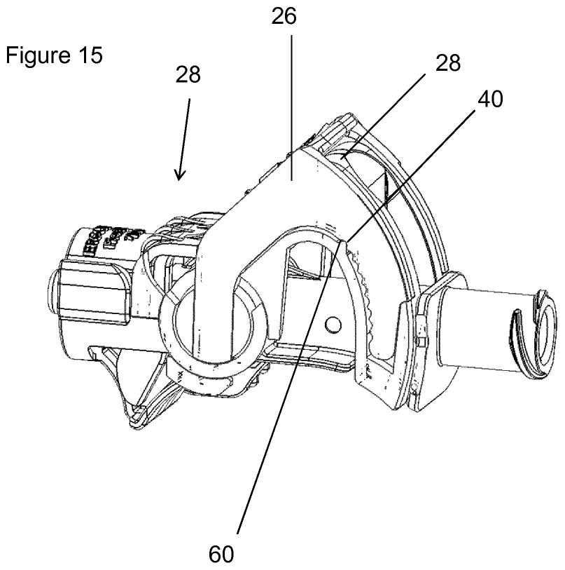

FIG. 14 shows a cross-sectional view of the fluid transfer device in the configuration seen in FIG. 13;

FIG. 15 shows a cut-away perspective view of the fluid transfer device in the configuration seen in FIG. 13;

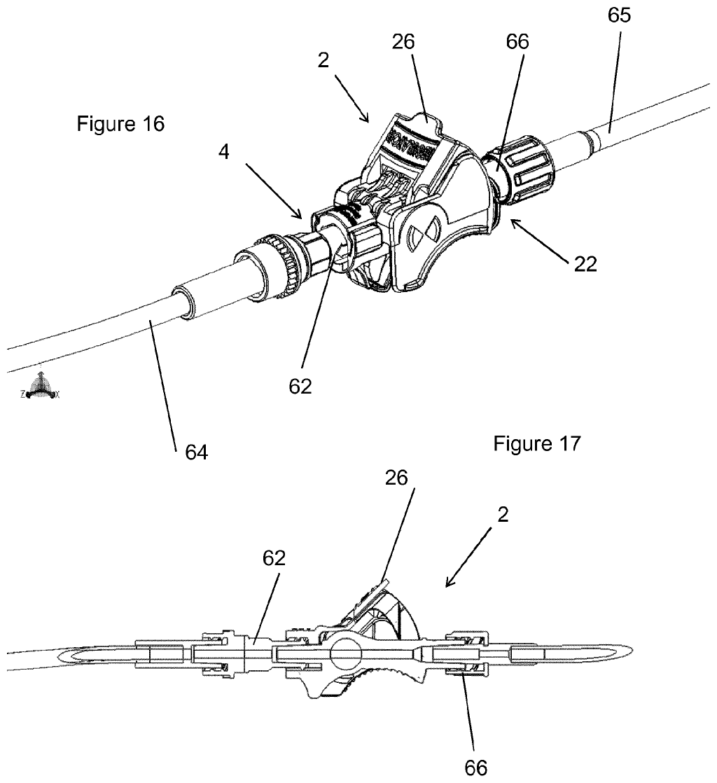

FIG. 16 shows a perspective view of the fluid transfer device in the configuration seen in FIG. 13 with a hub attached thereto;

FIG. 17 shows a cross-sectional view through the fluid transfer device with the hub attached thereto;

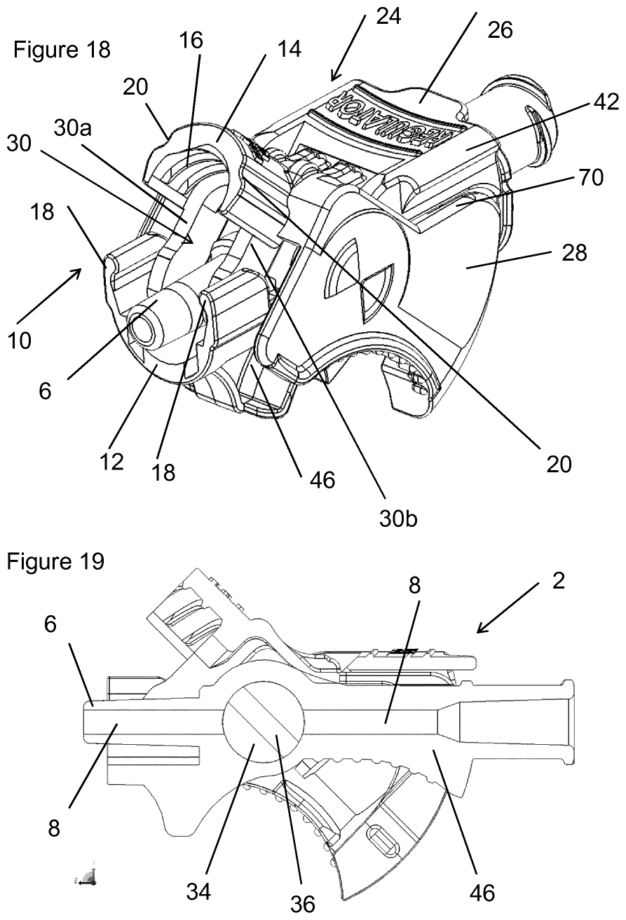

FIG. 18 shows a perspective view of the fluid transfer device in its final configuration;

FIG. 19 shows a cross-sectional view of the fluid transfer device in the final configuration seen in FIG. 18;

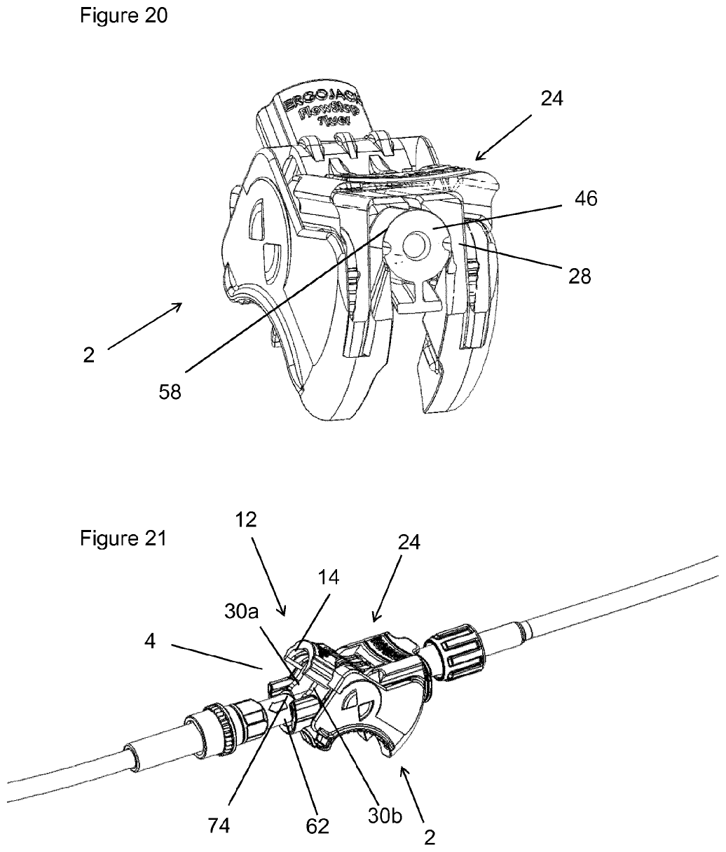

FIG. 20 shows a partially cut-away perspective view from the rear of the fluid transfer device in the configuration seen in FIG. 18;

FIG. 21 shows a perspective view of the fluid transfer device in the final configuration with a hub being disconnected therefrom;

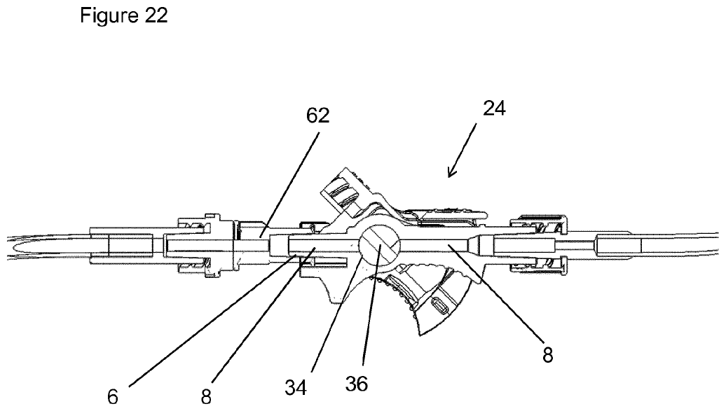

FIG. 22 shows a cross sectional view of the fluid transfer device seen in FIG. 21;

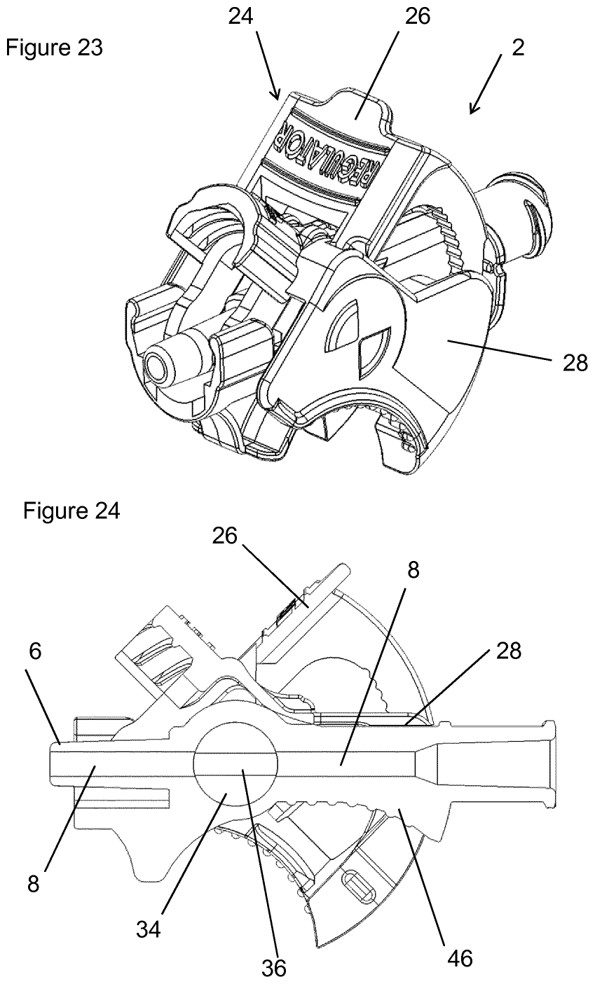

FIG. 23 shows a perspective view of the fluid transfer device;

FIG. 24 shows a cross sectional view of the fluid transfer device in the configuration seen in FIG. 23.

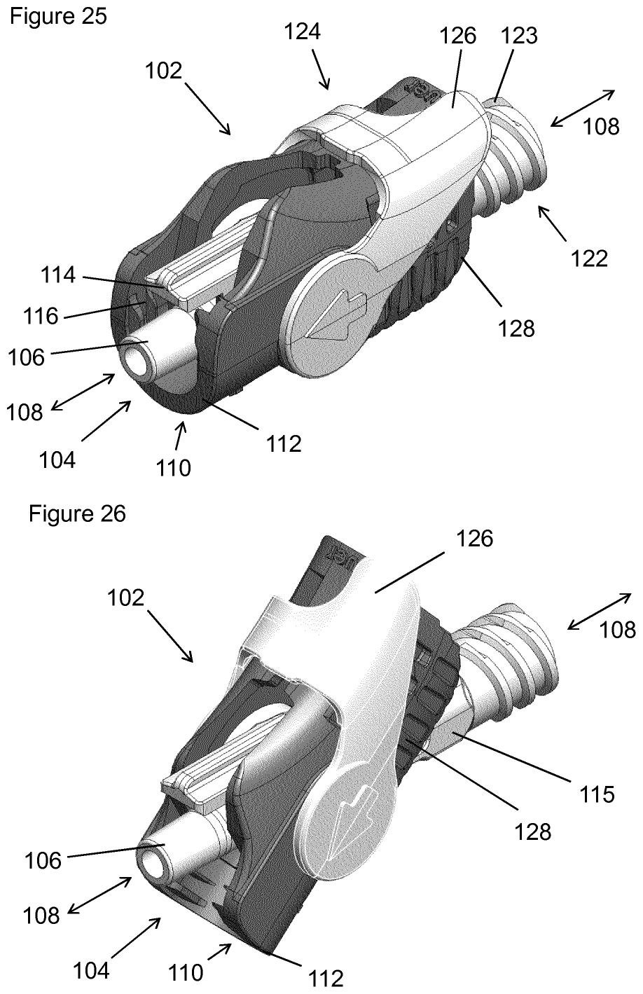

FIG. 25 shows a perspective view of a fluid transfer device in the form of a connector in accordance with a further embodiment of the present invention;

FIG. 26 shows a perspective view of the fluid transfer device of FIG. 25 with the disconnection member moved partially through its travel;

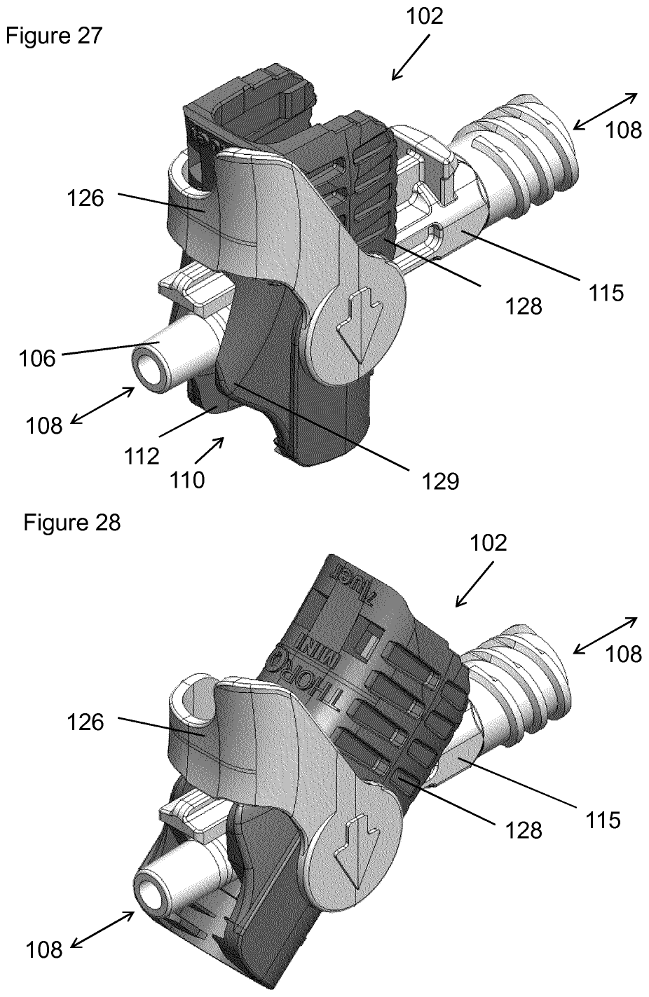

FIG. 27 shows a perspective view of the fluid transfer device of FIGS. 25 and 26 with the disconnection member in its final configuration;

FIG. 28 shows a perspective view of the fluid transfer device of FIGS. 25 to 27 with the disconnection member moved partially through its travel and the control member in its final configuration;

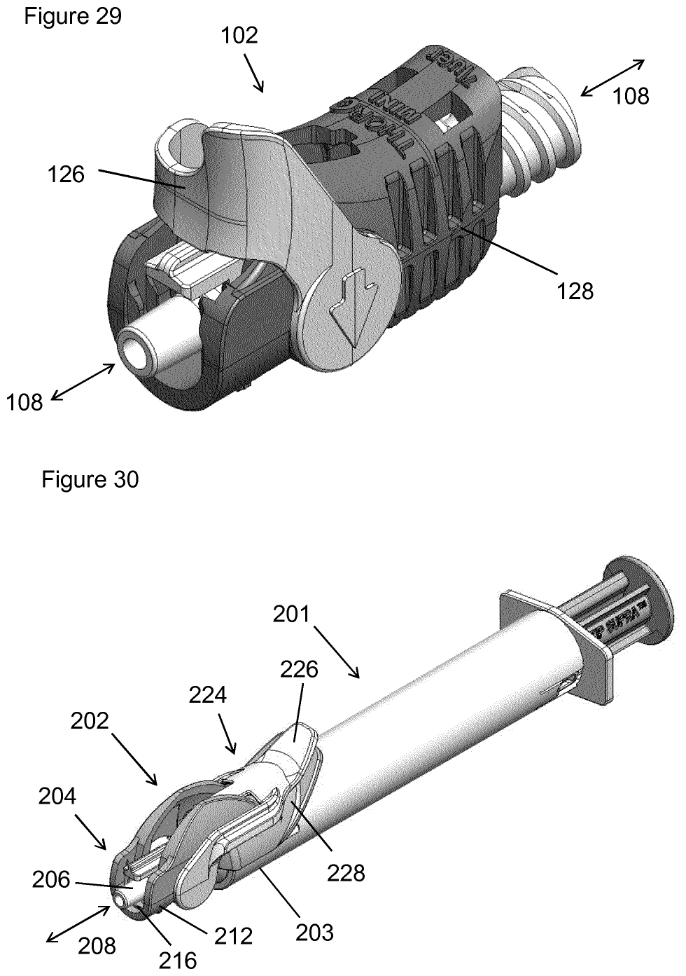

FIG. 29 shows a perspective view of the fluid transfer device of FIGS. 25 to 28 with the disconnection member in its initial configuration and the control member in its final configuration;

FIG. 30 shows a perspective view of a fluid transfer device in the form of a syringe in accordance with a further embodiment of the present invention;

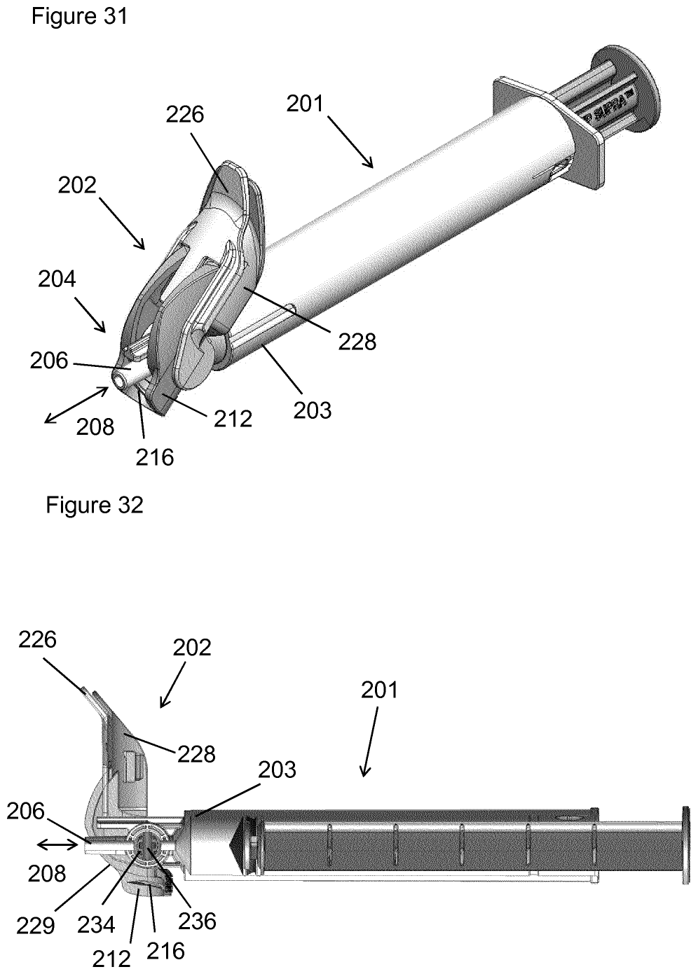

FIG. 31 shows a perspective view of the fluid transfer device of FIG. 30 with the disconnection member moved partially through its travel;

FIG. 32 shows a cross sectional view of the fluid transfer device of FIGS. 30 and 31 with the disconnection member in its final configuration;

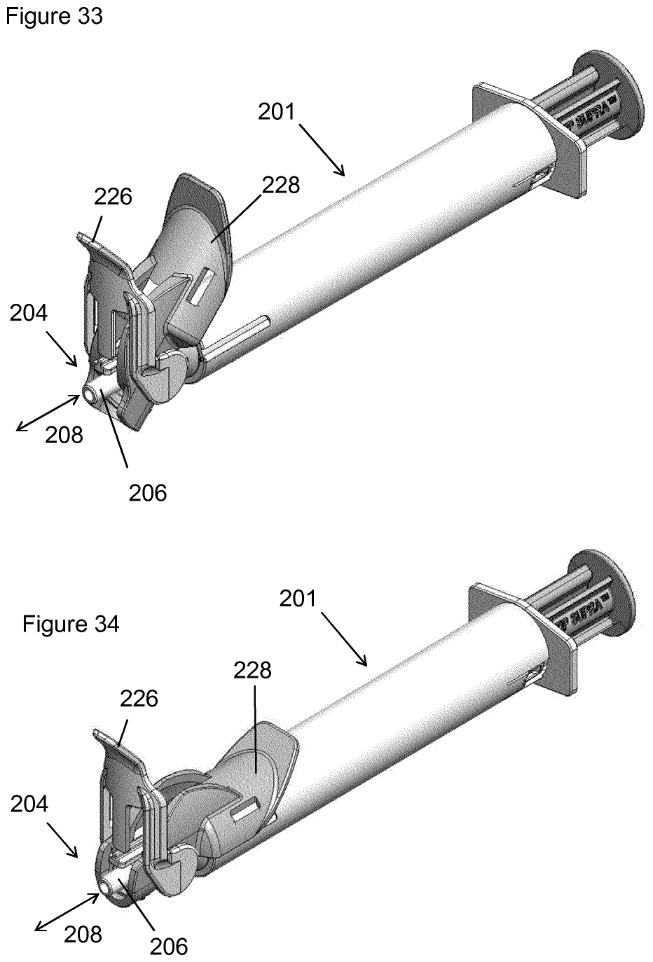

FIG. 33 shows a perspective view of the fluid transfer device of FIGS. 30 to 32 with the disconnection member partially through its travel and the control member in its final configuration; and

FIG. 34 shows the fluid transfer device of FIGS. 30-33 with the disconnection member in its initial configuration and the control member in its final configuration.

FIG. 1 shows a perspective view of a fluid transfer device in the form of a connector 2 in accordance with an embodiment of the present invention. A forward end of the connector 2 comprises a connector part 4 for connecting, in use, a corresponding hub. The connector part 4 comprises a fluid transfer tip in the form of a tapered tip 6 having a fluid flow path 8 extending therethrough. The connector part 4 further comprises a collar 10 surrounding the tapered tip 6. In this embodiment, the collar 10 extends 360° around the tapered tip 6 and is split into a first, lower, segment 12 and a second, upper, segment 14. The first segment 12 extends from a main body of the connector 2 (seen more clearly in FIG. 2) and is thus fixed relative to the tapered tip 6. The second segment 14 is moveably mounted relative to the tapered tip 6. The second segment 14 comprises an engagement feature in the form of internal threaded portion 16 arranged to engage, in use, with a corresponding engagement feature on a hub attached to the connector part 4. The first segment 12 further comprises a latch feature 18 at each of its extremes arranged to latch onto a corresponding detent 20 on the extremities of the second segment 14. This latching serves to hold the collar 10 in the closed configuration seen in FIG. 1.

The connector 2 further comprises a second connector part 22 arranged at the rear of the connector 2, for connection to a further component, e.g. a fluid transfer hose. The connector part 22 comprises an external thread 23 for engagement with an appropriately threaded further component.

The connector 2 further comprises a flow regulation mechanism 24 which comprises a flow control member 26 and a disconnection member 28. In this embodiment, each of the flow control member 26 and disconnection member 28 are in the form of pivotally mounted lever members. The second segment 14, of the collar 10, is integrally provided with the disconnection member 28 such that when the disconnection member 28 is moved, the second segment 14 moves.

The flow regulation mechanism 24 is shown in its initial configuration in FIG. 1. In this initial configuration the second segment 14 is closed around the tapered tip 6 such that the collar 10 has a closed configuration. The control member 26 is also pivoted forwards with respect to the disconnection member 28 such that the fluid flow path through the connector 2 is blocked. This can be seen more clearly in FIG. 8.

Whilst not shown in this Figure, a suitable hub may be connected to the connector part 4 and a further component, e.g. fluid transfer hose may be attached to the second connector part 22. In this particular embodiment, the connector part 4 conforms to the ISO 80369-7 standard, i.e. a Luer connector part. Of course, the connector part 4 may take any suitable form, and may instead conform to another of the ISO 80369 standards for example.

FIG. 2 shows a perspective view of the disconnection member 28. It can be seen more clearly from this Figure that the second segment 14 of the collar 12, seen in FIG. 1, is integrally provided with the disconnection member 28. The disconnection member 28 further comprises a fork extension 30 comprising a first leg 30a and second leg 30b. Referring back to FIG. 1, each of the first leg 30a and second leg 30b straddle the fluid transfer tip 6. The fork extension 30 is provided to interact with, and release, a hub from the connector part 4, when the disconnection member 28 is operated, i.e. pivoted.

FIG. 3 shows a perspective view of the flow control member 26. The flow control member 26 comprises first and second drive legs 32a, 32b which are engaged with a valve member 34. The valve member 34 comprises valve flow path 36 extending therethrough. Operation of the valve member 34 will be described in more detail below. The valve member 34 is mounted within the connector such that it can rotate about the axis labelled A-A. As will be appreciated by those skilled in the art, the engagement of the flow control member 26 with the rotatably mounted valve member 34, results in the flow control member 26 being pivotally mounted with its pivot axis also extending through the axis labelled A-A.

The flow control member 26 further comprises two position control arms 38. Each position control arm 38 comprises a series of detents 40. The flow control member 26 further comprises wing portions 42 which extend outward from an upper surface 44 of the flow control member 26. These wing portions 42 are dimensioned to engage with the disconnection member 28, seen in FIG. 1, when the flow control member 26 has been moved from its initial position, seen in FIG. 1, to an intermediate position in which it is in contact with the disconnection member.

FIG. 4 shows a perspective view of a main body 46 of the connector 2 seen in FIG. 1. The main body 46 comprises the tapered tip 6 and first segment 12 of the collar 10 seen in FIG. 1. The main body 46 further comprises the second connector part 22. The main body 46 provides the fluid flow path 8 extending through to the tapered tip 6. A cylindrical cavity 48 extending perpendicular to the fluid flow path 8, divides the fluid flow path 8 and allows the valve member 34, seen in FIG. 3, to be inserted into the fluid flow path 8. The main body 46 further comprises a recess 50 on each side for receiving a protrusion on the disconnection member 28 (not shown), in order to hold the disconnection member 28 in position.

FIG. 5 shows the flow control member 28 and associated valve member 34 assembled together with the main body 46.

FIG. 6 shows a perspective view of the rear of the disconnection member 28. First 52 and second (not visible) protrusions are provided on an inside surface 54 of the disconnection member 28. The protrusions 52 are positioned to engage with the recesses 50 on the main body 46 seen in FIG. 4 so as to hold the disconnection member 28 in a fixed position relative to the main body 46. The disconnection member 28 further defines a channel 56 between its two sides, above the inside surface 54 where the protrusions 52 are located. The channel 56 is narrower than a portion of the main body 46, around which the disconnection member 28 pivots. As a result, when the disconnection member 28 is pivoted, the main body 46 will pass into this channel 56 and cause deformation of the disconnection member 28, particularly in the region proximal to the channel 56. In this embodiment, the disconnection member 28 is made from a resilient material, such as a resilient plastic. As a result of this deformation, the disconnection member 28 will become resiliently biased, in this instance, back towards its initial position seen in FIG. 1. At the end of the channel 56 there is a circular opening 58 which has a diameter equal to that of the main body 46. When the disconnection member 28 is pivoted by an amount such that the main body 46 is received by the circular opening 58, the disconnection member 28 will no longer be deformed, and thus will no longer be resiliently biased. Therefore, once in this position, the disconnection member 28 will be stable and not move unless acted on by a user.

FIG. 7 shows a cut-away perspective view of the rear of the connector 2. In the configuration seen in FIG. 7, the protrusions 52 on the disconnection member 28 engage with the recesses 50 on the main body 46. This acts to hold the disconnection member 28 in the position seen in FIG. 1. As will be appreciated, in order to move the disconnection member, e.g. to disconnect a hub connected to the connector 2, the engagement between the protrusions 52 and recesses 50 must first be overcome. This may be achieved by applying a sufficiently large downward force to the disconnection member 28 such that the disconnection member 28 deforms to disengage the protrusions 52 and recesses 50. Once disengaged, the disconnection member 28 may then be pivoted. This Figure also more clearly shows how the channel 56 has a smaller dimension than the main body 46 in order to cause the deformation of the disconnection member 28 as it is pivoted out of its initial position seen in FIG. 1.

FIG. 8 shows a cross sectional view through the connector 2 with the flow regulation mechanism 24 in the initial configuration seen in FIG. 1. As can be seen, the flow control member 26 is in its forwardmost, initial position, and the disconnection member 28 is in its initial position wherein the collar 12 has a closed configuration. In this configuration, the valve member 34 is oriented such that the valve flow path 36 is misaligned with respect to the flow path 8 through the tapered tip 6 and the main body 46. In this initial configuration, fluid is completely prevented from flowing through the connector 2.

FIG. 9 shows a partially cut-away view of the connector 2. In this view, the side portion of the disconnection member 28 is not shown in order to show how the flow control member 26 interacts with the disconnection member 28 in order to hold it in a fixed position. The disconnection member 28 comprises a catch 60 arranged to engage with the detents 40 provided on the position control arms 38 of the flow control member 26 and shown in FIG. 3. As will be appreciated by those skilled in the art, the catch 60 may engage with any one of the series of detents 40 on the position control arms 38. Engagement between the catch 60 and any one of the detents 40 will hold the flow control member 26 in a fixed position relative to the disconnection member 28, resulting in the flow through the connector 2 being held at a stable flow rate.

Whilst not shown, the connector 2 may comprise a series of markings on an outer surface thereof indicative of the flow rate through the connector 2. The flow control member 26 may be aligned with at least one of these markings in order to set the flow rate through the connector 2.

FIG. 10 shows a perspective view of the connector 2, with the flow regulation mechanism 24 moved partially through its travel from its initial configuration to its final configuration. Specifically, the flow control member 24 has been pivoted partially towards the disconnecting member 28. The result of this pivotal movement can be seen in FIG. 11 which shows a cross-sectional view through the connector 2. With the flow control member 24 pivoted towards the disconnection member 28, the valve member 34 has been rotated such that the valve flow path 36 is partially aligned with the flow path 8 extending through the tapered tip 6 and the main body 46. As will be appreciated by those skilled in the art, with an appropriate hub attached to the connector part 4, and an appropriate fluid source connected to the second connector part 24, fluid may flow at a set flow rate from the fluid source through the connector 2. As will be appreciated by those skilled in the art, the flow rate permitted by the valve member 34 will be less than the flow rate if the valve flow path 36 were to be perfectly aligned with the flow path 8.

FIG. 12 shows a partially cut-away view of the connector 2, similar to the view seen in FIG. 9. The detents 40 on the position control arms 38 engage with the catch 60 on the disconnection member 28 and as a result the flow control member 26 is held in a fixed position relative to the disconnection member 28. As the disconnection member 28, is held in its position through engagement of the protrusions 52 and recesses 52, provided on the main body 50, as discussed above with respect to FIG. 7, the flow control member 26 is therefore also held in a fixed position relative to the main body 46. As a result, the valve member 34 is also held in a fixed position relative to the main body 46, and thus the flow rate through the connector 2 remains stable.

FIG. 13 shows a perspective view of the connector 2 with the flow regulation mechanism 24 moved further through its travel from its initial configuration towards its final configuration. In the position shown in FIG. 13, the flow control member 26 has been pivoted further towards the disconnection member 28, in order to further increase the flow through the connector 2. The disconnection member 28 remains in its initial, position, i.e. where the collar 12 is in the closed configuration.

FIG. 14 shows a cross sectional view through the connector 2 in the configuration seen in FIG. 13. When the flow control member 26 has been pivoted to the point just as it comes into contact with the disconnection member 28, in this embodiment, the valve member 34 is rotated to a position whereby the valve flow path 36 is completely aligned with the flow path 8 extending through the tapered tip 6 and the main body 46. Accordingly, fluid will be able to freely flow through the connector up to a maximum flow rate which is limited by the inner dimensions of the flow paths 8, 36.

FIG. 15 shows a partially cut-away view of the connector 2, in the configuration seen in FIGS. 13 and 14. The flow control member 26 is held in a fixed position relative to the disconnection member 28 by engagement between the protrusion 60 and the last detent 40. As a result, the flow control member 26 may remain in this position allowing fluid to flow through the connector 2, without further interact from a user, until a user wishes to adjust the flow rate.

FIG. 16 shows a perspective view of the connector 2, in the configuration seen in FIGS. 13-15, with a hub 62, from which a fluid hose 64 extends, connected to the connector part 4, and a second hub 66, from which a second fluid hose 66 extends, connected to the second connector part 22 The fluid hose 64 may, for example, be connected intravenously to a patient, and the second fluid hose 66 may be connected, for example, to a saline drip. Fluid from the saline solution may pass through the second fluid hose 66, the connector 2 and the fluid hose 64 into the patient. The fluid flow rate may be adjusted by a user operating the flow control member 24. FIG. 17 shows a cross sectional view through the connector 2 with the first and second hubs 62, 64 connected as seen in FIG. 16. With the first and second hubs 62, 64, along with their respective first and second fluid hoses 64, 68, attached to the connector 2, and with the flow control member 24 in the position shown, fluid can freely flow through the connector 2.

FIG. 18 shows a perspective view of the connector 2 with the flow regulation mechanism 24 moved through all of its travel to its final configuration. Starting from the position seen in FIG. 13, as the flow control member 26 is further depressed by a user, the force applied will be transferred to the disconnection member 28, via the wing portions 42 which transfer the force to a top edge 70 of the disconnection member 28. A user must first apply a threshold force to disengage the engagement between the protrusions 52 and recesses 50 (not seen in this Figure), in order to allow the disconnection member 28 to pivot. Additionally, in order to pivot the disconnection member 28, as the second segment 14 of the collar 12, which is integrally provided with the disconnection member 28, is latched to the first segment 12 via the latching of the latch feature 18 and detents 20, sufficient force must also be applied to overcome this latching, before pivotal movement of the disconnection member 28 can be achieved. Once the threshold force has been applied and the protrusions 52 and recesses 50 have been disengaged, and the latching has been overcome, further force applied by the user to the flow control member 28 will cause the disconnection member 28 to pivot relative to the main body 46. Of course, in order to pivot the disconnection member 28 relative to the main body 46, sufficient force must be applied to also overcome the resilient bias generated as a result of deformation of the disconnection member 28, as discussed previously.

As the disconnection member 28 is pivoted towards the final configuration seen in FIG. 18, the pivotal movement of the disconnection member 28 moves the second segment 14 away from the tapered tip 6, thereby releasing any engagement between the internal thread 16 and the hub attached to the connector 2. Additionally, as the disconnection member 28 is pivoted, the forked section 30 also pivots relative to the fluid transfer tip 6, and the first and second legs 30a, 30b of the forked section 30, are pivoted upwards and move, at least partially, along the tapered tip 6. When a hub is attached to the connector 2, and a friction fitting is achieved with the tapered tip 6, the movement of the first and second legs 30a, 30b will function to push the hub off the tapered tip 6 and release the friction fitting.

As the flow regulation mechanism 24 is moved through its travel into the final configuration seen in FIG. 18, in order to release a hub from the connector 2, the movement of the disconnection member 28 by the flow control member 26 also moves the valve member 34 (not visible in FIG. 18). In this embodiment, as the flow regulation mechanism 24 is moved into the final configuration seen in FIG. 18, the valve member 34 is moved to a position which completely stops the flow of fluid through the connector as shown in FIG. 19. Advantageously, this means that when the hub is disconnected from the connector 2, the fluid flow through the connector 2 is prevented and so no fluid escapes. This allows for a dry disconnection of an attached hub. As discussed previously, this may be advantageous for a number of reasons, for example, it may prevent the wastage of expensive drugs which may be contained within the fluid, it may also prevent contamination of surrounding work surfaces with the fluid.

As mentioned above, FIG. 19 shows a cross-sectional view through the connector 2, in the final configuration seen in FIG. 18. The valve member 34 has been rotated further such that the valve flow path 36 is completely misaligned with the fluid flow path 8 through the tapered tip 6 and the main body 46. As a result, fluid cannot pass through the connector 2.

When a user releases their applied force, the flow regulation mechanism 24 will be held in the final configuration seen in FIG. 18. FIG. 20 shows a partially cut-away view from the rear of the connector 2. With the flow regulation mechanism 24 in the configuration seen in FIG. 18, the main body 46 rests in the circular opening 58 on the disconnection member 28. As a result, the disconnection member 28 is not deformed, thus does not experience any resilient bias and remains in the position seen in the final configuration.

FIG. 21 shows a perspective view of the connector 2 in the configuration seen in FIG. 18 as the hub 62 is disconnected from the connector part 4. As the flow regulation mechanism 24 is moved into this final configuration, the second segment 14 is moved away from the hub to move the collar 12 into the open configuration 12. Additionally, as the flow regulation mechanism 24 is moved, the first and second legs 30a, 30b push against the rear surface 74 of the hub 62, thereby advancing the hub 62 along the tapered tip 6 (not visible in this Figure), thus releasing the friction fitting between the hub 62 and the tapered tip 6.

FIG. 22 shows a cross-sectional view of the arrangement seen in FIG. 21. With the flow regulation mechanism 24 in its final configuration, the valve flow path 36, on the valve 34, is misaligned with the flow path 8 through the tapered tip 6, and the flow path 48, on the main body 46, when the hub 62 becomes released from the tapered tip 6. In this embodiment, the valve member 34 is configured such that the valve flow path 36 becomes misaligned before the hub 62 is completely released, this ensures that little, or no, fluid can escape the connector 2 follow release of the hub 62.

FIG. 23 shows a perspective view of the connector 2, with the flow regulation mechanism 24 moved back out of its final configuration through its travel towards its initial configuration. The disconnection member 28 has remained stationary, but the flow control member 26 has been pivoted away from the disconnection member 28. Pivoting the flow control member 26 away from the disconnection member 28 moves the valve member 34 (not visible in this Figure) back to a position which allows fluid to pass through the connector 2.

FIG. 24 shows a cross-sectional view through the connector 2 in the configuration seen in FIG. 23. The disconnection member 28 remains in its position whereby it is pivoted towards the main body 46. Pivoting the flow control member 26 back towards the initial configuration rotates the valve member 34 to a position wherein the valve flow path 28 is brought into alignment with the fluid flow path 8 through the tip 6 and the main body 46. This configuration seen in FIGS. 23 and 24 advantageously allows a user to release a hub from the connector 2, and subsequently allow fluid to flow through the connector 2 without reattaching a hub e.g. to flush the device.

As will be appreciated by those skilled in the art, the flow regulation mechanism 24, comprising the flow control member 26 and disconnection member 28 may be operated without a hub attached to the connector 2. This may, for example, allow a user to draw fluid through the connector 2 before being attached to a hub. Additionally, the connector 2 may be operated so as to be in the final configuration seen in FIG. 18 prior to attaching a hub. Moving the flow regulation mechanism into this final configuration, and thus moving the second segment 14 away from the fluid transfer tip 6 may allow a hub to be more easily attached to the connector 2. With the second segment 14 in the open configuration, a hub may be slid onto the connector part 4, and then the disconnection member 28 may be released from its final configuration position, seen in FIG. 18, and its resilient bias caused by deformation of the disconnection member 28 may drive the disconnection member back to its position seen in 13, and thus bring the second segment 14 back towards the first segment 12 and thus form a collar 10 having a closed configuration. In this position, the internal threads 16 on the second segment 14 may not perfectly align with the corresponding engagement features on the hub, and so the hub may be rotated by a small amount until they are aligned and the hub is positively engaged by the internal threads 16 on the second segment 14 of the collar 10.

FIGS. 25-29 show perspective views of a fluid transfer device in the form of a connector 102 in accordance with a further embodiment of the present invention. A forward end of the connector 102 comprises a medical connector part 104 for connecting, in use, a corresponding hub (e.g. a hub 62 as seen in FIGS. 16 and 17). The connector part 104 comprises a fluid transfer tip in the form of a tapered tip 106 having a fluid flow path 108 extending therethrough. The connector part 104 further comprises a collar 110 substantially surrounding the tapered tip 106. The collar 110 is split into a first, lower, segment 112 and a second, upper, segment 114. The second segment 114 extends from a main body 115 of the connector 102 (seen in FIG. 27) and is thus fixed relative to the tapered tip 106, which also extends from the body 115. The first segment 112 comprises an engagement feature in the form of an internal groove or threaded portion 116 arranged to engage, in use, with a corresponding engagement feature on a hub attached to the connector part 104.

The body 115 further comprises a second connector part 122 arranged at the rear of the connector 102, for connection to a further component, e.g. a fluid transfer hose. The second connector part 122 comprises an external thread 123 for engagement with an appropriately threaded further component.

The connector 102 further comprises a flow regulation mechanism 124 which comprises a flow control member 126 and a disconnection member 128. In this embodiment, each of the flow control member 126 and disconnection member 128 are in the form of pivotally mounted lever members that are independently operable. The control member 126 comprises a valve member (not shown) similar to that discussed above with reference to FIG. 3 and similarly arranged to control the flow of fluid through the fluid flow path 108 of the connector 102. Thus, pivoting the control member 126 causes the valve member to rotate, which serves to open or close the flow path 108 through the body 115, thereby controlling the flow rate of fluid. The first segment 112, of the collar 110, is integrally provided with the disconnection member 128 such that when the disconnection member 128 is moved, the first segment 112 moves relative to the body 115.

The flow regulation mechanism 124 is shown in an initial configuration in FIG. 25. In this initial configuration the first segment 112 is close to the tapered tip 106 such that the collar 110 has a closed configuration. The control member 126 and disconnection member 128 are both pivoted to be parallel to the flow path 108 such that the flow path 108 through the connector 102 is fully unrestricted. The control member 126 and the disconnection member 128 are arranged such that the control member 126 is positioned to rest on an upper surface of the disconnection member 128.

Whilst not shown in this Figure, a suitable hub may be connected to the connector part 104 and a further component, e.g. a fluid transfer hose may be attached to the second connector part 122. In this particular embodiment, the connector part 104 conforms to the ISO 80369-7 standard, i.e. a Luer connector part. This means the connector part 104 forms a friction fitting with a corresponding Luer hub, in use. Of course, the connector part 104 may take any suitable form, and may instead conform to another of the ISO 80369 standards for example.

FIG. 26 shows the connector 102 of FIG. 25 with the disconnection member 128 moved partially through its travel from its initial configuration towards its final configuration. Thus, the disconnection member 128 has pivoted to be inclined to the axis of the fluid flow path 108. As a result, the first segment 112 has been pivoted away from the tapered tip 106 such that the collar 110 is in a partially open configuration. This means that the threaded portion or groove 116 is no longer engaged with a corresponding engagement feature on the hub and the hub is no longer locked.

The pivoting of the disconnection member 128 pushes the control member 126 such that the control member 126 also pivots to an inclined position with respect to the axis of the fluid flow path 108. In the configuration shown in FIG. 26, the control member 126 is positioned such that the valve member partially restricts the flow through the connector 102.

FIG. 27 shows the connector 102 of FIGS. 25 and 26 with the disconnection member 128, and thus the control member 126, in its final configuration. In this configuration, the disconnection member 128 and the control member 126 have pivoted to be substantially perpendicular to the axis of the fluid flow path 108. As a result, the first segment 112 has been pivoted further away from the tapered tip 106 such that the collar 110 is in a fully open configuration. In order to fully release the hub from its friction fitting with the connector 102, the disconnection member 128 comprises a shoulder 129 that pushes along the tapered tip 106 during the final stages of travel towards the final configuration, in order to advance the hub along the tip 106 and release its friction fitting.

In this final configuration, the control member 126 is arranged perpendicular to the flow path 108. Thus, the valve member of the control member 126 is arranged to fully block the flow through the connector 102. Thus, in a similar manner to the embodiments described above, the connector 102 is capable of dry disconnection of an attached hub.

FIG. 28 shows the connector 102 of FIGS. 25 to 27 with the disconnection member 128 partially through its travel between its initial configuration and its final configuration and the control member 126 in its final configuration (i.e. with the fluid flow path 108 fully closed). As the disconnection member 128 is arranged to push the control member 126 only when moving from its initial configuration to its final configuration (i.e. not in the reverse direction), it will be appreciated that the control member 126 and the disconnection member 128 may advantageously be operated separately.

For example, FIG. 29 shows the connector 102 of FIGS. 25 to 28 with the disconnection member 128 in its initial configuration in which, in use, a hub may be connected to the connector 102, and the control member 126 in its final configuration (i.e. with the fluid flow path 108 fully closed). Thus, the flow of fluid 108 through the connector 102 may be controlled by pivoting the control member 126 alone, without having to actuate the disconnection member 128. It will be appreciated that the control member 126 may be pivoted to any intermediate position between its initial configuration and its final configuration while the disconnection member 128 is in its initial configuration, thereby controlling the flow rate while a hub is connected to the connector part 104. The shoulder 129 of the disconnection member 128 only comes into contact to push the hub when the disconnection member 128 moves into its final configuration.

Although the above embodiments described the fluid transfer device of the present invention as a connector part, the Applicant has appreciated that the device may form an integral part of a further device to which a hub is to be connected.

FIGS. 30 to 34 show perspective views of a fluid transfer device in the form of a syringe 201 in accordance with a further embodiment of the present invention. The syringe 201 comprises a connector mechanism 202 that is essentially the same as the connector 102 described above with reference to FIGS. 25 to 29, except that the connector mechanism 202 is formed at a front end of the syringe body 203 and therefore does not comprise a second connector part arranged at the rear of the connector mechanism 202 for connection to a further component. Instead, the rear of the connector mechanism 202 is arranged with the body 203 of the syringe 201 such that a flow path 208 is established from an integral fluid chamber in the body 203 of the syringe 201 through the connector mechanism 202, and connector part 204. As before, the connector part 204 comprises a fluid transfer tip 206 for connection, in use, with a corresponding hub (e.g. a needle hub).

The connector mechanism 202 comprises a flow regulation mechanism 224, which comprises a flow control member 226, and a disconnection member 228. The flow control member 226 comprises a valve member 234 (shown in FIG. 32) arranged to selectively open and close the fluid flow path 208 to control the flow from the syringe 201 to a connected hub. The disconnection member 228 is integrally provided with a moveable segment 212 that forms part of a collar extending around the fluid transfer tip 206. As before, the moveable segment 212 includes an engagement feature on its inner cylindrical surface in the form of a circumferential groove or angled thread 216.

Operation of the flow regulation mechanism 224 is the same as the operation of the flow regulation mechanism 124 described above. Thus, actuation of the flow regulation mechanism 224 and the disconnection member 228 serves to respectively control the flow through the hub and to release the hub in the same way as described above with reference to FIGS. 25 to 29. The flow regulation mechanism 224 and the disconnection member 228 are independently operable.

In FIG. 30, the flow control member 226 and the disconnection member 228 are in an initial configuration in which the control member 226 and the disconnection member 228 are parallel to the flow path 208 such that the flow path 208 through the connector mechanism 202 is fully unrestricted.

FIG. 31 shows the syringe 201 of FIG. 30 with the disconnection member 228 moved partially through its travel from its initial configuration towards its final configuration. Thus, the disconnection member 228 has been pivoted to be inclined to the axis of the fluid flow path 208. As described above, when a hub is connected to the syringe 201 in use, the pivoting of the disconnection member 228 acts pivot the collar segment 212 away from the fluid transfer tip 206 and unlock a connected hub.

The pivoting of the disconnection member 228 pushes the control member 226 such that the control member 226 also pivots to an inclined position with respect to the axis of the fluid flow path 208. This pivoting of the control member 226 causes the valve member to rotate. In the configuration shown in FIG. 31, the control member 226 is positioned such that the valve member partially restricts the flow through the connector mechanism 202.

FIG. 32 shows a cross-sectional side view of the syringe 201 of FIGS. 30 and 31 with the disconnection member 228, and thus the control member 226, in its final configuration in which the disconnection member 228 and the control member 226 are perpendicular to the axis of the fluid flow path 208. In this configuration, the disconnection member 228 is arranged to fully release a hub from the connector mechanism 202. As well as disengaging the thread 216, movement of the disconnection member 228 to its final configuration causes a shoulder 229 to push forwards along the tip 206 and thereby release the friction fitting with a hub.

The valve member 234 is shown in FIG. 32. The valve member 234 defines a fluid flow path 236 that is pivoted with the movement of flow control member 226. In the initial configuration of the flow control member 226 (as shown in FIG. 30), the fluid flow path 236 of the valve member 234 is aligned with the fluid flow path 208. Thus, in the initial configuration, fluid may flow through the fluid flow path 208 from an integral fluid chamber within the body 203 of the syringe 201, through the connection mechanism 202 to a connected hub. However, in the final configuration of the flow control member 226 (as shown in FIG. 32), the fluid flow path 236 of the control member 226 is perpendicular to the fluid flow path 208. Thus, the valve member 234 is arranged to fully prevent the flow of fluid between the body 203 of the syringe 201 and a connected hub. Thus, in a similar manner to the embodiments described above, the connector mechanism 202 is capable of dry disconnection of an attached hub.

FIG. 33 shows the syringe 201 of FIGS. 30 to 32 with the disconnection member 228 partially through its travel between its initial configuration and its final configuration and the control member 226 in its final configuration (i.e. with the fluid flow path 208 fully closed). As the disconnection member 228 is arranged to push the control member 226 only when moving from its initial configuration to its final configuration (i.e. not in the reverse direction), it will be appreciated that the control member 226 and the disconnection member 228 may advantageously be operated independently of one another.

For example, FIG. 34 shows the syringe 201 of FIGS. 30 to 33 with the disconnection member 228 in its initial configuration in which, in use, a hub may be attached to the tip 206 of the syringe 201, and the control member 226 in its final configuration (i.e. with the fluid flow path 208 fully closed). Thus, the flow of fluid through the fluid flow path 208 may be controlled by pivoting the control member 226 alone, without having to actuate the disconnection member 228. It will be appreciated that the control member 226 may be pivoted to any intermediate position between its initial configuration and its final configuration while the disconnection member 228 is in its initial configuration, thereby controlling the flow rate while a hub is connected to the tip 206.

What is claimed is:

1. A fluid transfer device comprising:
a body;
a medical connector part, extending from the main body, for connection, in use, with a corresponding hub;
a fluid flow path extending through the body and medical connector part;
a flow regulation valve arranged in the fluid flow path in the body, for selectively controlling the flow rate of fluid through the fluid flow path;
a disconnection member mounted to the body and arranged to move relative to the medical connector part to release, in use, the hub connected to the medical connector part; and
wherein the flow regulation valve and disconnection member are independently operable.

2-6. (canceled)

7. The fluid transfer device as claimed in claim 1, wherein the disconnection member is moveable relative to the medical connector part from an initial connection position in which, in use, a corresponding hub may be connected to the medical connector part, to a final disconnection position in which, in use, the disconnection member acts to release the hub connected to the medical connector part.

8. The fluid transfer device as claimed in claim 7, wherein the movement of the disconnection member towards its final disconnection position is arranged to drive movement of the flow regulation valve towards its a final configuration, such that the flow regulation valve and the disconnection member move together in unison.

9. The fluid transfer device as claimed in claim 1, wherein the disconnection member is independently movable between an initial connection position and a final disconnection position while the flow regulation valve is in a final configuration.

10. The fluid transfer device as claimed in claim 1, wherein the medical connector part comprises a fluid transfer tip.

11. The fluid transfer device as claimed in claim 10, wherein the fluid transfer tip is tapered and the disconnection member is arranged to release an attached hub by moving at least partially along the tapered tip in order to advance the hub along the tapered tip and release a friction fitting.

12. The fluid transfer device as claimed in claim 10, wherein the disconnection member comprises a shoulder arranged to move forwards along the tapered tip only when the disconnection member moves towards a final disconnection position.

13. The fluid transfer device as claimed in claim 10, wherein the medical connector part comprises a collar extending at least partially around the fluid transfer tip and arranged in use to positively engage with a hub.

14. The fluid transfer device as claimed in claim 13, wherein the collar comprises a first segment and a second segment, wherein the second segment is arranged to be moved by the disconnection member from an initial engagement position, in which it is arranged to engage, in use, with the hub attached to the connector part, to a final disengagement position in which it is disengaged from the hub.

15. The fluid transfer device as claimed in claim 14, wherein the second segment of the collar is integrally provided with the disconnection member.

16. The fluid transfer device as claimed in claim 14, wherein the second segment of the collar comprises an internally threaded portion.

17. The fluid transfer device as claimed in claim 1, further comprising an integral fluid chamber in fluid communication with the medical connector part.

18. The fluid transfer device as claimed in claim 1, wherein the disconnection member comprises a pivotally mounted lever member.

19. The fluid transfer device as claimed in claim 1, further comprising a flow control member, comprising a pivotally mounted lever member, arranged to directly operate the flow regulation valve.

20. (canceled)

21. The fluid transfer device as claimed in claim 19, wherein the disconnection member is arranged beneath the flow control member such that movement of the disconnection member towards its/a final disconnection position always drives movement of the flow control member so as to cut off the flow rate of fluid through the fluid flow path.

22. A medical fluid transfer device comprising:
a connector part for connecting, in use, a corresponding hub;
a fluid flow path extending through the connector part; and
a flow regulation mechanism, for selectively controlling a fluid flow through the fluid flow path, wherein the flow regulation mechanism has a travel from an initial configuration to a final configuration and wherein a first portion of the travel at least partially opens the fluid flow path and wherein a second portion of the travel closes the fluid flow path and releases, in use, a corresponding hub attached to the connector part.

23. The medical fluid transfer device as claimed in claim 22 wherein, in the second portion of travel, once the flow regulation mechanism has fully closed the fluid flow path, the flow regulation mechanism then releases the hub attached to the connector part.

24-28. (canceled)

29. The medical fluid transfer device as claimed in claim 22, wherein the flow regulation mechanism comprises a control member arranged to control the fluid flow through the fluid flow path and a disconnection member arranged to release, in use, a corresponding hub attached to the connector part, and wherein the flow regulation mechanism is arranged such that across the first portion of travel only the control member is moved from an initial control position to an intermediate control position and wherein, across the second portion of travel, both the control member and disconnection member are moveable respectively towards a final control position and a final disconnection position.

30. (canceled)

31. The A-medical fluid transfer device as claimed in claim 29, wherein the control member is arranged to drive movement of the disconnection member during the second portion of travel, such that the control member and disconnection member move together in unison.

32-39. (canceled)

40. The A-medical fluid transfer device as claimed in claim 22, wherein the connector part comprises a fluid transfer tip and a collar, the collar extending at least partially around the fluid transfer tip and arranged in use to positively engage with a hub, wherein the collar comprises a first segment and second segment, wherein the second segment is arranged to be moved by the flow regulation mechanism from an initial position, in which it is arranged to engage, in use, with the hub attached to the connector part, to a final position in which it is disengaged from the hub.

41-47. (canceled)
